(19) United States
(12) Patent Application Publication
(10) Pub. No.: US 2023/0086229 A1
(43) Pub. Date: Mar. 23, 2023

(54) METHOD FOR DETERMINING A DIAGNOSTICALLY RELEVANT SECTIONAL PLANE

(71) Applicant: Siemens Healthcare GmbH, Erlangen (DE)

(72) Inventors: Daniel GIESE, Erlangen (DE); Jens WETZL, Spardorf (DE); Michaela SCHMIDT, Uttenreuth (DE)

(73) Assignee: Siemens Healthcare GmbH, Erlangen (DE)

(21) Appl. No.: 17/948,409

(22) Filed: Sep. 20, 2022

(30) Foreign Application Priority Data
Sep. 22, 2021 (EP) 21198221.0

(51) Int. Cl. G06T 7/73; A61B 5/055; G06T 7/00

(52) U.S. Cl. CPC G06T 7/74; A61B 5/055; G06T 7/0012; G06T 2207/10088; G06T 2207/30048; G06T 2207/20084

(57) ABSTRACT

A computer-implemented method for determining an orientation of at least one diagnostically relevant sectional plane for heart imaging in a three-dimensional magnetic resonance imaging image dataset, comprises: providing the three-dimensional image dataset; applying a trained function to the three-dimensional image dataset to determine a position of at least one landmark; determining the orientation of the at least one diagnostically relevant sectional plane as a function of at least one landmark; and providing the orientation of the at least one diagnostically relevant sectional plane.

### CROSS-REFERENCE TO RELATED APPLICATION(S)

The present application claims priority under 35 U.S.C. § 119 to European Patent Application No. 21198221.0, filed Sep. 22, 2021, the entire contents of which are incorporated herein by reference.

### FIELD

One or more example embodiments of the present invention relate to a computer-implemented method for determining an orientation of at least one diagnostically-relevant sectional plane for heart imaging in a three-dimensional magnetic resonance imaging image dataset and a determining system, which is designed to carry out the method. One or more example embodiments of the present invention also relate to a magnetic resonance imaging system, to a computer program product and to a computer-readable storage medium. One or more example embodiments of the present relate, moreover, to a computer-implemented method for providing a trained function for determining a position of at least one landmark in a three-dimensional magnetic resonance imaging image dataset.

### BACKGROUND

In magnetic resonance imaging (acronym: MRI) a two-dimensional sectional image of a sectional plane of an examination object can be recorded with an MRI system. The sectional plane can have any orientation relative to the examination object or relative to the MRI system. The examination object can be a person, in particular a patient, or an animal. Typical orientations of a sectional plane are coronal, sagittal and axial. These orientations are also referred to as “standard orientations”.

The examination object is positioned in a scan region of the MRI system for recording the sectional image. A magnetic field is applied inside the scan region. The magnetic field is composed of a homogeneous basic magnetic field and a magnetic field gradient. The proton spins of the examination object in the scan region orient themselves along this magnetic field. The strength of the magnetic field determines a Larmor frequency at which the spins precess. By irradiating an excitation pulse, exactly those spins are exciting whose Larmor frequency matches the frequency of the excitation pulse. The excited spins are tilted relative to the magnetic field and precess in phase. The spins relax again as a function of the material surrounding the excited spins. The spins orient themselves along the magnetic field again and dephase. The duration until the spins are oriented again and the duration of the dephasing depend on the material surrounding the spins. An electromagnetic signal emitted by the spins can be acquired on relaxation. The sectional image or three-dimensional MRI image data can be reconstructed from this signal.

The magnetic field gradient serves for spatial encoding. The magnetic field strength is different at different locations of the examination object within the scan region due to the magnetic field gradient. The spins thereby also precess at a different Larmor frequency as a function of their position in the magnetic field or in the examination object. By way of suitable selection of the frequency of the excitation pulse, only particular spins can be excited, therefore. In this way, spins can be excited in any sectional plane as a function of the orientation of the magnetic field gradient and the frequency of the excitation pulse. The spins excited with the excitation pulse, namely the spins in the sectional plane, all precess at approximately the same frequency and are arranged inside an approximately constant region of the magnetic field gradient. The acquired signal thereby describes the relaxation of the spins in the sectional plane. The sectional image of the sectional plane can be reconstructed in this way. A three-dimensional image dataset can be reconstructed by (parallel) recording of more than one sectional image.

The heart is oriented differently in different examination objects. To be able to carry out a diagnosis or examination in respect of the heart, a sectional image of the heart has to comprise or map a particular view of the heart. In particular, the sectional image has to map a diagnostically relevant sectional plane of the heart. The orientation of the diagnostically relevant sectional plane is typically specified by or dependent on a position of at least one landmark in the heart. Due to the individual orientation of the heart it is not possible to record the sectional image of the heart using one of the standard orientations (coronal, sagittal, axial).

It is known that an orientation of a diagnostically relevant sectional plane for a sectional image is determined iteratively by an experienced medical operator. For this, firstly a first plurality of two-dimensional sectional images is recorded, for example in accordance with the standard orientations. The medical operator can determine an orientation of one or more further sectional planes on the basis of this first plurality of sectional images. From these sectional planes, sectional images are in turn recorded, which in turn serve for determining an orientation of one or more further sectional plane(s) from which sectional images are recorded. In particular, at least one landmark can be determined by the medical operator in each recorded sectional image. The orientation of the following sectional planes or of the sectional planes of the next iteration can be determined on the basis of this at least one landmark or its position in the sectional image. In this way, the sectional plane, which comprises at least one landmark, which in turn specifies the orientation of the diagnostically relevant sectional plane, can be iteratively determined. The diagnostically relevant sectional plane or its orientation can then be derived from this sectional plane. One possible embodiment of this iterative method is described for example by Maier et al. In “Kardiale Magnetresonanztomographie—Anatomie and Planung” [Cardiac Magnetic Resonance Tomography—Anatomy and Planning], Journal for Kardiologie—Austrian Journal of Cardiology 2003; 10: 3-7.

Blansit et al. describe in “Deep Learning-based Prescription of Cardiac MRI Planes”, Radiology: Artificial Intelligence 2019; 1(6):e180069 a method for determining the landmarks in the two-dimensional sectional images via a trained function. The above-described iterative method can be automated in this way.

Nevertheless, it is necessary both in the manual iterative method and in the automated iterative method to record a large number of sectional images, which are not diagnostically relevant and serve merely for determining an orientation of the heart in the examination object and for deriving the diagnostically relevant sectional plane therefrom.

The described iterative method for determining the at least one diagnostically relevant sectional plane is very time-consuming. The iterative method is thus also very cost-intensive. The examination object has to be positioned in the typically narrow scan region of the MRI system so as to remain still or as immobile as possible in order to record the individual sectional images. In addition, it can be necessary that the examination object has to hold their breath for each sectional image. This results in a high level of discomfort for the examination object as early as in the run-up to the actual examination, or the recording of the sectional image of the diagnostically relevant sectional plane.

### SUMMARY

It is therefore the object of one or more example embodiments of the present invention to provide a method, which enables fast and automatic determining of a diagnostically relevant sectional plane.

The object is achieved by a computer-implemented method for determining an orientation of at least one diagnostically-relevant sectional plane for heart imaging in a three-dimensional magnetic resonance imaging image dataset, by a computer-implemented method for providing a trained function for determining a position of at least one landmark in a three-dimensional magnetic resonance imaging image dataset, by a determining system, by a magnetic resonance imaging system, by a computer program product and by a computer-readable storage medium in accordance with the independent claims. Advantageous developments are set out in the dependent claims and in the following description.

The inventive solution to the object, according to one or more example embodiments of the present invention, will be described hereinafter in relation to both claimed apparatuses and in relation to the claimed method. Features, advantages or alternative embodiments mentioned in this connection should also likewise be transferred to the other claimed subject matters, and vice versa. In other words, the concrete claims (which are directed for example to an apparatus) can also be developed with the features, which are described or claimed in connection with a method. The corresponding functional features of the method will be developed by corresponding concrete modules.

Furthermore, the inventive solution to the object, according to one or more example embodiments of the present invention, will be described in relation to both methods and apparatuses for determining an orientation of at least one diagnostically-relevant sectional plane for heart imaging in a three-dimensional magnetic resonance imaging image dataset as well as in relation to methods and apparatuses for providing a trained function. Features and alternative embodiments of data structures and/or functions in methods and apparatuses for determining can be transferred here to analogous data structures and/or functions in methods and apparatuses for adjusting/optimizing/training. Analogous data structures can be identified in particular here by the use of the prefix “training”. Furthermore, the trained functions used in method and apparatuses for determining an orientation of at least one diagnostically-relevant sectional plane for heart imaging in a three-dimensional magnetic resonance imaging image dataset can have been trained or adjusted and/or provided in particular by methods for providing the trained function.

One or more example embodiments of the present invention relate to a computer-implemented method for determining an orientation of at least one diagnostically relevant sectional plane for heart imaging in a three-dimensional magnetic resonance imaging image dataset. The method comprises a method step of providing the three-dimensional image dataset. The method also comprises a method step of applying a trained function to the three-dimensional image dataset. A position of at least one landmark is determined in the process. The method comprises a method step of determining the orientation of the at least one diagnostically relevant sectional plane as a function of at least one landmark. The method comprises a method step of providing the orientation of the at least one diagnostically relevant sectional plane.

The three-dimensional magnetic resonance imaging (acronym: MRI) image dataset maps at least one part of an examination object. The examination object is a person, in particular a patient, or an animal. The mapped part of the examination object comprises at least one part of the heart of the examination object. The three-dimensional MRI image dataset (hereinafter also referred to as a three-dimensional image dataset) or the three-dimensional MRI scan comprises a three-dimensional mapping of the heart or at least of the part of the heart.

The three-dimensional image dataset comprises a plurality of voxels, which are arranged in a three-dimensional voxel matrix. A position in the examination object can be assigned to each voxel. Each voxel comprises a voxel value, which represents an intensity value, which is derived from a signal emitted from the corresponding position of the examination object. The intensity value can specify a gray scale value or a color value of the voxel in a representation of the three-dimensional image dataset.

In embodiments of the present invention, the three-dimensional image dataset can also be a two-dimensional multi-slice scan or a multi-slice 2D scan. A plurality of two-dimensional slice images or sectional images of the heart or of the part of the heart can have been spatially sequentially recorded in different planes. The two-dimensional sectional images are oriented parallel to each other. The two-dimensional sectional images of the multi-slice scan can be arranged “stacked”, arranged one behind the other, therefore. The “stacked” two-dimensional sectional images jointly form a “pseudo” three-dimensional mapping of the heart or of the part of the heart. Each slice image comprises a plurality of pixels, which are arranged in a two-dimensional pixel matrix. A pixel value, which is formed analogously to the above-described voxel value, is assigned to each pixel. Parts of the following description, which refer to the voxels or voxel values of the three-dimensional image dataset, can be transferred analogously to the pixels or pixel values of the two-dimensional multi-slice scan.

The three-dimensional image dataset can be an overview scan of the part of the examination object.

In other words, the three-dimensional image dataset can have been recorded with a fast scan protocol. In particular, the scan protocol can be optimized in respect of a scan duration for recording the overview scan. For this, an image quality, for example a spatial resolution and/or a signal-to-noise ratio, can be reduced in favor of a shorter scanning time. In particular, a spatial resolution of the three-dimensional image dataset can be too low for a diagnostic evaluation. In particular, the resolution is sufficient to be able to determine the at least one landmark or its position in the three-dimensional image dataset.

The diagnostically relevant sectional plane or slice plane is a two-dimensional sectional plane through the three-dimensional image dataset. The orientation of the diagnostically relevant sectional plane can be determined relative to the three-dimensional image dataset. In particular, the orientation relative to an MRI system, with which the three-dimensional image dataset was recorded, can be determined. In other words, the orientation of the diagnostically relevant sectional plane can be based on a coordinate system of the MRI system. The orientation describes the spatial location or the spatial orientation of the diagnostically relevant sectional plane. In other words, the orientation can describe an angulation of the diagnostically relevant sectional plane. In particular, the spatial location can be described for example by a direction of a normal vector on the diagnostically relevant sectional plane. In embodiments of the present invention, the orientation can also describe a position of the diagnostically relevant sectional plane. The position indicates where the diagnostically relevant sectional plane should intersect the examination object, or which points should lie in the diagnostically relevant sectional plane. In other words, the orientation comprises information about the spatial orientation or spatial location of the diagnostically relevant sectional plane and in embodiments of the present invention, information about the position of the diagnostically relevant sectional plane.

“Diagnostically relevant” means that, based on a two-dimensional sectional image or slice image of the diagnostically relevant sectional plane, a diagnosis can be made for the examination object or an examination can be carried out. The sectional image maps a section through the examination object in the diagnostically relevant sectional plane. In particular, the sectional image can map a predefined or standardized cross-section of the heart of the examination object. In other words, the orientation of the diagnostically relevant sectional plane is selected in such a way that the corresponding sectional image maps the predefined or standardized cross-section of the heart. The diagnosis can be for example a diagnosis of myocarditis or a pericardial effusion or a cardiac infarct or a tumor or a disease of a heart valve, etc.

The three-dimensional image dataset can in particular be received or recorded or retrieved in the method step of providing the three-dimensional image dataset.

The three-dimensional image dataset can be received or retrieved in particular via an interface. The three-dimensional image dataset can be retrieved or received for example from a database. The database can be an image database of a medical facility for example of a hospital. The database can in particular be a Picture Archiving and Communication System, acronym: PACS. The database can be stored on a server. The server can be a local server or a Cloud server. The three-dimensional image dataset can thus be retrieved or received or provided from/by the server in the method step of providing the three-dimensional image dataset.

Alternatively, the three-dimensional image dataset can be recorded in the method step of providing the three-dimensional image dataset. In other words, in the method step a signal can be acquired with the MRI system, from which the three-dimensional image dataset can be reconstructed. The signal can be based, as described, on a relaxation of spins in the mapped part of the examination object.

In the method step of applying the trained function, the trained function is applied to the three-dimensional image dataset in order to determine the position of the at least one landmark.

In particular, in each case a position can be determined for more than one landmark. The landmark describes an anatomy of the heart. The landmark can be for example a heart valve or a venous or arterial entrance or exit of the heart or the apex, etc. The position of the landmark can be described in the voxel matrix by the position of the at least one voxel, which maps the landmark in the at least one three-dimensional image dataset. In particular, the position of the landmark in the examination object can be inferred from the position of the voxel.

In the following the designation “the at least one landmark” always also comprises the position of the at least one landmark.

In the method step of applying the trained function, the position of the at least one landmark is generated via the trained function on the basis of the three-dimensional image data.

Generally, a trained function imitates cognitive functions, which connect humans to human thought. In particular, the trained function can be adjusted to new circumstances and identify and extrapolate patterns by way of training based on training data.

Generally, parameters of a trained function can be adjusted via training. In particular, supervised training, semi-supervised training, unsupervised training, reinforcement learning and/or active learning can be used for this. Furthermore, representation learning (an alternative term is “feature learning”) can be used. In particular, the parameters of the trained functions can be iteratively adjusted by a plurality of training steps.

In particular, a trained function can comprise a neural network, a support vector machine, a decision tree and/or a Bayesian network, and/or the trained function can be based on k-means clustering, Q-learning, genetic algorithms and/or association rules. In particular, a trained function can comprise a combination of a plurality of uncorrelated decision trees or an ensemble of decision trees (random forest). In particular, the trained function can be determined via XGBoosting (eXtreme Gradient Boosting). In particular, a neural network can be a deep neural network, a convolutional neural network or a convolutional deep neural network. Furthermore, a neural network can be an adversarial network, a deep adversarial network and/or a generative adversarial network. In particular, a neural network can be a recurrent neural network. In particular, a recurrent neural network can be a network with long-short-term-memory (LSTM), in particular a Gated Recurrent Unit (GRU). In particular, a trained function can comprise a combination of the described approaches. In particular, the approaches described here are cited for a trained function network architecture of the trained function.

In the method step of determining the orientation of the at least one diagnostically relevant sectional plane, the orientation of the diagnostically relevant sectional plane is determined as a function of at least one landmark. In other words, the orientation of the at least one diagnostically relevant sectional plane is determined as a function of position of the at least one landmark.

In particular, the orientation of more than one diagnostically relevant sectional plane can be determined in the method step.

In particular, the orientation of the at least one diagnostically relevant sectional plane can be determined as a function of more than one landmark or their positions.

For example, the orientation of the diagnostically relevant sectional plane can be specified by the positions of three or more landmarks. The diagnostically relevant sectional plane can be oriented in such a way that the three or more landmarks lie in the sectional plane.

Alternatively, the orientation of the diagnostically relevant sectional plane can depend on the position of one or two landmark(s). The diagnostically relevant sectional plane can be oriented in such a way that the one or two landmark(s) lie in the sectional plane. The orientation can then also depend on prior knowledge, for example of a standard orientation (coronal, sagittal or axial).

Alternatively, the orientation of the diagnostically relevant sectional plane can be specified by an orientation relative to the at least one landmark. For example, the diagnostically relevant sectional plane can be perpendicular to a plane spanned by three landmarks.

As described above, the orientation indicates the orientation of the diagnostically relevant sectional plane relative to the three-dimensional image dataset or to the MRI system.

In the method step of providing the orientation of the at least one diagnostically relevant sectional plane, the orientation is provided in particular via an interface.

The orientation can be indicated for example as a normal vector on the at least one diagnostically relevant sectional plane in a coordinate system of the MRI system. Alternatively, or in addition, the orientation can be indicated in such a way that a gradient control unit can be actuated for generating a magnetic field gradient. The gradient control unit can actuate at least one gradient coil in such a way that a magnetic field gradient is generated, which is perpendicular to the at least one diagnostically relevant sectional plane. The magnetic field gradient can be designed in such a way that a magnetic field with a predefined, constant magnetic field strength is applied in the plane of the at least one diagnostically relevant sectional plane.

The inventors have found that all relevant landmarks can be determined at once in the three-dimensional image dataset with a trained function. It is thereby sufficient to record a scan of the examination object in advance for planning the at least one diagnostically relevant sectional plane or for determining its orientation. An iterative method is thus no longer necessary for determining the orientation of the at least one diagnostically relevant sectional plane. In this way, the comfort of the examination object can be increased since the method for determining the diagnostically relevant sectional plane can be sped up and only one scan is necessary for planning the diagnostically relevant sectional plane. This also results in a cost saving. In particular, a spatial connection between different landmarks can be determined by determining all landmarks in a three-dimensional image dataset since the positions of all landmarks can be determined in a single three-dimensional image dataset. This could not be ensured in previous methods since there the positions were determined in different, spatially unconnected two-dimensional image datasets. Error propagation due to an iterative method can be avoided by way of the inventive method.

According to one aspect of the method, the three-dimensional image dataset maps at least one part of a heart. The three-dimensional image dataset is an overview scan of the heart.

The three-dimensional image dataset maps at least one part of the heart of the examination object. In particular, the three-dimensional image dataset can map the entire heart of the examination object.

The overview scan is designed as described above. In particular, a lower image quality, in particular a higher level of noise and/or a reduced spatial resolution, is accepted in the overview scan for faster recording of the three-dimensional image dataset or a shorter scan duration.

In particular, the overview scan can also be a two-dimensional multi-slice scan, which can be formed as described above.

The inventors have found that an overview scan of the heart or of the part of the heart is sufficient in order to determine the position of the at least one landmark. In particular, a lower image quality, in particular a higher level of noise and/or a lower spatial resolution, is tolerable in the three-dimensional image dataset since the three-dimensional image dataset is not recorded in order to make a diagnosis or an examination. In this way, the time for recording the three-dimensional image dataset can be minimized and the method for determining the orientation of the at least one diagnostically relevant sectional plane can be sped up.

According to a further aspect of embodiments of the present invention, the at least one landmark is one of the following landmarks: apex, mitral valve, aortic valve, pulmonary valve, tricuspid valve.

In particular, the position of the landmark can relate to or describe or mark a striking point of the apex, the mitral valve, the aortic valve, the pulmonary valve or the tricuspid valve.

In embodiments of the present invention, the at least one landmark can be one of the following landmarks:

left ventricular apex (acronym: LV apex)
right ventricular apex (acronym: RV apex)
right ventricle extent, acronym: RV extent)
mitral valve, midpoint (acronym: MV midpoint)
aortic valve, midpoint (acronym: AV midpoint)
tricuspid valve, midpoint (acronym: TV midpoint)
pulmonary valve, midpoint (acronym: PA center point)
aortic arch
descending aorta
four points on the vessel wall of the aortic bulb (anterior, posterior, left, right)
four points on the vessel wall of the pulmonary artery (left, right, superior, inferior) at a suitable location for flow measurement of the pulmonary artery (pulmonary artery flow plane)
four points on the annulus of the pulmonary valve (left, right, superior, inferior) (pulmonary valve insertion)
four points on the annulus of the mitral valve (septal, lateral, superior, inferior) (mitral valve insertion)
four points on the annulus of the tricuspid valve (septal, lateral, superior, inferior) (tricuspid valve insertion)
right ventricle insertion points (anterior, posterior) on the left ventricle
descending aorta centerline
ascending aorta centerline.

The inventors have found that at least one predefined or a standardized landmark can be determined. In this way, the orientation of the at least one diagnostically relevant sectional plane can be derived from the position of the at least one landmark from a known spatial relation. This can ensure a comparability between different examination objects and/or between sectional images of an examination object, which were recorded at different instants. If the orientation of the at least one diagnostically relevant sectional plane is based on at least one predefined or standardized landmark or its position, the orientation can be determined according to a standard method. In particular, the orientation of a standardized diagnostically relevant sectional plane can then be determined.

According to a further aspect of embodiments of the present invention, the at least one diagnostically relevant sectional plane is one of the following sectional planes: four chamber plane, three chamber plane, two chamber plane, vertical long axis, horizontal long axis, short axis.

The location of said planes relative to each other is described in particular in Maier et al., “Kardiale Magnetresonanztomographie—Anatomie and Planung”, Journal for Kardiologie—Austrian Journal of Cardiology 2003; 10: 3-7.

In embodiments of the present invention, the at least one diagnostically relevant sectional plane can be one of the following sectional planes:

vertical long axis (acronym: VLA)
horizontal long axis (acronym: HLA)
three chamber view (acronym: 3CH)
four chamber view (acronym: 4CH)
short axis (acronym: SAX)
right ventricle two chamber view (acronym: RV-2CH)
right ventricle three chamber view (acronym: RV-3CH)

### Aortic Flow Plane

left ventricular outflow tract plane (acronym: LVOT)
mitral valve plane (acronym: MV Plane).

The orientation of the sectional planes can be derived from or depend on the landmarks, or their positions indicated below in brackets after the sectional planes:

VLA (LV apex, MV midpoint+prior knowledge: parasagittal)
HLA (LV apex, MV midpoint+prior knowledge: orthogonal to VLA)
3CH (LV apex, MV midpoint, AV midpoint)
4CH (LV apex, MV midpoint, RV extent)
SAX (midpoint of LV apex and MV midpoint, RV extent+prior knowledge: orthogonal to VLA and HLA)
RV-2CH (RV apex, TV midpoint+prior knowledge: parasagittal)
RV-3CH (RV apex, TV midpoint, PA center point)
Aortic Flow Plane (Aortic bulb (anterior), Aortic bulb (posterior), Aortic bulb (left), Aortic bulb (right))
LVOT (Aortic bulb (anterior), Aortic bulb (posterior)+prior knowledge: slice in the midpoint of the two landmarks, orthogonal to the connecting vector of the landmarks)
MV Plane (MV insertion (septal), MV insertion (lateral), MV insertion (superior), MV insertion (inferior)).

The inventors have found that the at least one diagnostically relevant sectional plane can be a predefined or standardized sectional plane. The inventors have found that a diagnosis and/or an examination can be standardized in this way. In particular, the medical operator, who makes the diagnosis on the basis of the sectional image of the diagnostically relevant sectional plane or carries out the examination on the basis of the sectional image of the diagnostically relevant sectional plane, can be familiar with the view in the diagnostically relevant sectional plane. A diagnosis can be standardized and sped up in this way.

According to a further aspect of embodiments of the present invention, in the method step of applying the trained function, the position of the at least one landmark is determined in the form of a probability distribution for the position of the at least one landmark in the three-dimensional image dataset.

The probability distribution can also be referred to as a “heatmap”.

In particular, a probability can be assigned to each voxel of the three-dimensional image dataset, and this indicates how probable it is that the at least one landmark is mapped in the corresponding voxel. In embodiments of the present invention, the probability can be a number between 0 and 1. A probability of “0” can mean that the at least one landmark is not mapped in the corresponding voxel and “1” can mean that the at least one landmark is certainly mapped in the voxel.

In particular, analogously to the three-dimensional image dataset the probability distribution can comprise a three-dimensional voxel matrix. Each voxel of this voxel matrix corresponds to a voxel of the three-dimensional image dataset. The voxels of both voxel matrices are identically arranged. Each voxel of the probability distribution comprises the above-described probability, corresponding to the voxel, as the voxel value.

If the position of more than one landmark is determined, a separate probability distribution can be determined for each landmark.

Alternatively, the probability distribution can be represented or described in respect of more than one landmark in a combined probability distribution.

The inventors have found that the probability distribution means uncertainties can be taken into account when determining the position of the at least one landmark. In addition, the orientation of the at least one diagnostically relevant sectional plane can to a certain extent be variable as a function of the probability distribution. In particular, the orientation of the diagnostically relevant sectional plane can be varied in such a way that, on average, the probability of the voxels, which lie in the sectional plane, of all landmarks on which the orientation depends, is maximal or minimal.

According to a further aspect of embodiments of the present invention, the method step of providing the orientation of the at least one diagnostically relevant sectional plane comprises a method step of providing at least one first scanning parameter value for controlling a medical-technical magnetic resonance imaging system for recording a two-dimensional sectional image of the at least one diagnostically relevant sectional plane.

In particular, the MRI system comprises at least one gradient coil, which can be actuated with a gradient control unit incorporated by the MRI system. In embodiments, the MRI system comprises more than one gradient coil. In particular, the one or more gradient coil(s) is/are incorporated by a gradient coil unit.

The at least one gradient coil can be actuated with the gradient control unit in such a way that the at least one gradient coil generates a magnetic field gradient. The magnetic field gradient is overlaid on a basic magnetic field in the scan region of the MRI system. The magnetic field inside of the scan region is therefore composed of the homogenous basic magnetic field and the magnetic field gradient.

In particular, the gradient control unit can be provided with the first scanning parameter value. The gradient control unit can actuate the at least one gradient coil on the basis of the first scanning parameter value. In particular, a strength and/or an orientation of the magnetic field gradient can be specified by the first scanning parameter value.

Advantageously, the magnetic field gradient or the orientation of the magnetic field gradient can be perpendicular to the of the at least one diagnostically relevant sectional plane. In particular, the magnetic field gradient is then constant in the at least one diagnostically relevant sectional plane. In particular, a strength of the magnetic field in the diagnostically relevant sectional plane is designed in such a way that the spins in the diagnostically relevant sectional plane precess at a Larmor frequency, which matches the frequency of the excitation pulse.

In particular, if the MRI system comprises more than one gradient coil, the first scanning parameter value can indicate which current strength is to be applied to which gradient coil so the magnetic field gradient is designed as described.

Alternatively, or in addition, the first scanning parameter value can indicate a “steepness” of the magnetic field gradient, which is to be generated with the at least one gradient coil. A slice thickness of a slice of the examination object mapped in the sectional image can be specified in this way. The sectional image maps a two-dimensional projection of the slice along its thickness. The “steeper” the magnetic field gradient, the thinner is the projected slice. The “flatter” the magnetic field gradient, the thicker is the projected slice.

Alternatively, or in addition, the first scanning parameter value can indicate a frequency of the excitation pulse. The frequency can depend on the strength of the magnetic field or of the magnetic field gradient in such a way that the excitation pulse excites exactly the spins in the diagnostically relevant sectional plane. In particular, the frequency of the excitation pulse thus also depends on the position of the diagnostically relevant sectional plane in the magnetic field.

The two-dimensional sectional image maps a section through the examination object in the at least one diagnostically relevant sectional plane. The two-dimensional sectional image can be recorded with the MRI system.

The inventors have found with a first scanning parameter value, the MRI system can be actuated in such a way that a two-dimensional sectional image of the diagnostically relevant sectional plane can be recorded. In particular, the orientation of the diagnostically relevant sectional plane can comprise the first scanning parameter value. In other words, the orientation of the diagnostically relevant sectional plane can be provided in such a way that the MRI system can be directly actuated.

According to a further aspect of embodiments of the present invention, the at least one first scanning parameter value is derived from the orientation of the at least one diagnostically relevant sectional plane.

In particular, the at least one first scanning parameter value is formed in such a way that the two-dimensional sectional image can be recorded in the at least one diagnostically relevant sectional plane as a function of the first scanning parameter value. In other words, the first scanning parameter value depends directly on the orientation of the at least one diagnostically relevant sectional plane.

In particular, the first scanning parameter value can be derived from the orientation in such a way that, on the basis of the first scanning parameter value, the gradient control unit can actuate the gradient coil in such a way that in the diagnostically relevant sectional plane the magnetic field is constant when recording the two-dimensional sectional image. In particular, the first scanning parameter value can be derived in such a way that the gradient control unit actuates the at least one gradient coil on the basis of the first scanning parameter value in such a way that a magnetic field gradient is oriented perpendicular to the diagnostically relevant sectional plane.

The inventors have found that on the basis of the orientation of the diagnostically relevant sectional plane, the first scanning parameter value can be determined with which the two-dimensional sectional image of the diagnostically relevant sectional plane can be recorded. Recording of the two-dimensional sectional image of the diagnostically relevant sectional plane, in particular actuating the MRI system for recording the two-dimensional sectional image, can thus depend directly on the orientation by way of the scanning parameter.

According to a further aspect of embodiments of the present invention, the method also comprises a method step of recording the two-dimensional sectional image with the magnetic resonance imaging system as a function of the at least one first scanning parameter value and a method step of providing the two-dimensional sectional image.

The examination object is arranged in the magnetic field when recording the two-dimensional sectional image. The magnetic field is composed of the homogeneous basic magnetic field and the above-described magnetic field gradient, which is overlaid on the basic magnetic field. The proton spins of the examination object are oriented along the magnetic field and precess at a Larmor frequency, which depends on a magnetic field strength at the site of the spins. The spins can be excited by irradiation of an excitation pulse. The spins, which precess at the frequency of the excitation pulse, are excited in the process. The excited spins precess in phase as a result of the excitation. In addition, the spins are tilted relative to the magnetic field during excitation. At the end of the excitation pulse the spins in the magnetic field relax as a function of a material that surrounds them. This means the spins dephase and orient themselves along the magnetic field again. The spins emit an electromagnetic signal in the process, which can be acquired by an antenna or coils. In particular, a speed of the relaxation is acquired. The speed depends on the material.

When recording the two-dimensional sectional image of the diagnostically relevant plane, the magnetic field gradient is adjusted with the first scanning parameter value in such a way that the spins in the diagnostically relevant sectional plane precess (at least approximately) at the frequency of the excitation pulse. In addition, when recording the two-dimensional sectional image, the electromagnetic signal, after excitation of the spins in the diagnostically relevant sectional plane, is read out or acquired on relaxation. In addition, when recording the two-dimensional sectional image, the two-dimensional sectional image is reconstructed from the acquired signal.

In the method step of providing the two-dimensional sectional image the two-dimensional sectional image can be provided by an interface.

In particular, the two-dimensional sectional image can be loaded into a PACS or stored there when providing the two-dimensional sectional image. In other words, the two-dimensional sectional image can be stored in a database.

Alternatively, or in addition, in the method step of providing the two-dimensional sectional image, the two-dimensional sectional image can be displayed to the medical operator. In particular, the sectional image can be displayed via a display unit, for example a screen or a monitor.

The inventors have found that the two-dimensional sectional image can be recorded and provided as a function of the orientation of the at least one diagnostically relevant sectional plane. In particular, the medical operator can be provided with the two-dimensional sectional image. In particular, the medical operator can make a diagnosis or an examination on the basis of the two-dimensional sectional image.

According to a further aspect of embodiments of the present invention, the method comprises a method step of determining an extent of a three-dimensional volume image perpendicular to the diagnostically relevant sectional plane. The three-dimensional volume image is spanned by the diagnostically relevant sectional plane and the extent. The method also comprises a method step of providing at least one second scanning parameter value for controlling a magnetic resonance imaging system for recording the three-dimensional volume image. The at least one second scanning parameter value is derived from the orientation of the at least one diagnostically relevant sectional plane and the extent. The method also comprises a method step of recording the three-dimensional volume image with the magnetic resonance imaging system as a function of the at least one second scanning parameter value. The method also comprises a method step of providing the three-dimensional volume image.

The three-dimensional volume image is a three-dimensional representation or mapping of the at least one part of the heart, which comprises the diagnostically relevant sectional plane. In other words, the two-dimensional sectional image of the diagnostically relevant sectional plane is incorporated by the three-dimensional volume image. The three-dimensional volume image has an extent perpendicular to the diagnostically relevant sectional plane. The diagnostically relevant sectional plane can be arranged at any position inside the three-dimensional volume image. The three-dimensional volume image is spanned by the diagnostically relevant sectional plane and the extent. In other words, the region of the heart, which is mapped in the three-dimensional volume image, is described by the diagnostically relevant sectional plane and the extent perpendicular to this sectional plane.

The three-dimensional volume image can map for example a characteristic curve of an inflow to or outflow from the heart. The three-dimensional volume image can in particular be designed to analyze a flow in the heart in the volume image.

In the method step of determining the extent, the extent of the three-dimensional volume image perpendicular to the diagnostically relevant sectional plane is determined. The extent can comprise for example 0.1 cm or 0.5 cm or 1 cm or 2 cm. The extent is determined in particular as a function of an anatomical actuality of the mapped heart. The anatomical actuality can be determined in particular as a function of the three-dimensional image dataset. In particular, the anatomical actuality can be determined or be specified by or depend on one or more landmark(s) as described above. For example, the three-dimensional volume image can be extended in such a way that it maps the entire aortic arch. The extent then depends on the size of the aortic arch perpendicular to the diagnostically relevant sectional plane. In the example, the diagnostically relevant sectional plane runs through the aortic arch. The dependency of other anatomies can be analogously transferred to the example.

In the method step of providing the at least one second scanning parameter value, the second scanning parameter value is analogously provided as described above for the first scanning parameter value. The second scanning parameter value can in particular be formed analogously to the first scanning parameter value.

In addition, the second scanning parameter value comprises a sequence of frequencies of the excitation pulse. The sequence of the frequencies is formed in such a way that the spins are excited in different sectional planes parallel to the diagnostically relevant sectional plane inside the region spanned by the three-dimensional volume image. In particular, one of the frequencies is designed in such a way that the spins are excited in the diagnostically relevant sectional plane.

Alternatively, the second scanning parameter value comprises a sequence of strengths of the magnetic field gradient. The sequence of strengths of the magnetic field gradient is designed in such a way that with a constant frequency of the excitation pulse, the spins can be excited in different sectional planes inside the three-dimensional volume image.

In the method step of recording the three-dimensional volume image, the three-dimensional volume image is recorded as a function of the second scanning parameter value with the magnetic resonance imaging system. The sequence of strengths of the magnetic field gradient acquires a signal for each frequency of the sequence of frequencies or for each strength of the magnetic field gradient. The three-dimensional volume image can be reconstructed as is known from the signals from the magnetic resonance imaging.

In the method step of providing the three-dimensional volume image, the three-dimensional volume image is provided as described above for the two-dimensional slice image.

The inventors have found that in some cases a two-dimensional mapping of the diagnostically relevant sectional plane is not sufficient for a diagnosis or examination. The inventors have found that starting from the diagnostically relevant sectional plane, an extent of a three-dimensional volume image can then be determined, which is suitable for the diagnosis or the examination.

According to a further aspect of embodiments of the present invention, the trained function is based on a neural convolutional network and/or a U-Network.

The neural convolutional network can also be referred to as a “convolutional neural network” (acronym: CNN). The U-Network can also be referred to as a “U-Net”.

The convolutional network or the U-Network comprise an input and an output. Input data can be input into the input, to which data the trained function or the convolutional network or the U-Network can be applied. The input data can comprise in particular the three-dimensional image dataset.

Output data is generated when the trained function is applied to the input data. The output data can comprise in particular the position of the at least one landmark, in particular the probability distribution.

The convolutional network or the U-Network comprise a plurality of layers. The layers are arranged in particular in an order. The input data passes through the convolutional network or the U-Network in the order. Each of the layers comprises a plurality of neurons or nodes. The neurons of the layers can be linked among themselves. In other words, a neuron can be linked to one or more neuron(s) of the preceding slice. In particular, the preceding neurons can influence a value of the linked neuron in the following slice. In particular, a link can be weighted. The weight of the link determines the influence, which the preceding neuron has on the neuron linked to the link in the following slice. The weights can be adjusted or learned or trained during training of the network or during training of the trained function.

The convolutional network or the U-Network comprise at least one input layer and one output layer.

The number of neurons incorporated by the input layer can in particular match the number of voxels in the voxel matrix of the three-dimensional image dataset.

In embodiments of the present invention, the output layer can comprise at least one neuron. Alternatively, the number of neurons in the output layer can match the number of voxels in the voxel matrix of the three-dimensional image dataset if the probability distribution is determined by applying the trained function. In embodiments of the present invention, a probability distribution can be determined for each landmark by applying the trained function. The number of neurons of the output layer can then match the product of the number of voxels in the voxel matrix of the three-dimensional image dataset and the number of landmarks.

The convolutional network can comprise one or more convolutional layer(s). The convolutional network can also comprise one or more deconvolutional layer(s). In particular, the one or more convolutional layer(s) can be arranged in the order before the one or more deconvolutional layer(s). The convolutional network can also comprise one or more pooling layer(s).

The U-Network can in particular comprise a convolutional network designed as described above. In particular, the layers of the convolutional network in the U-Network can be linked together in a u-shaped manner. In other words, in the U-Network the input layer is linked to the output layer. The slice which follows the input layer is linked to the last but one slice, to the slice before the output layer therefore, etc. Via the link, information from a slice can be taken into account in the slice linked to this slice.

The inventors have found that a convolutional network or a U-Network are especially suitable for determining the position of the at least one landmark in the three-dimensional image data. In particular, a convolutional network or a U-Network is especially suitable for determining the probability distribution of the at least one landmark.

One or more example embodiments of the present invention also relate to a computer-implemented method for providing a trained function for determining a position of at least one landmark in a three-dimensional magnetic resonance imaging image dataset.

The method comprises a method step of receiving at least one three-dimensional training image dataset. The method also comprises a method step of receiving at least one annotated three-dimensional training image dataset. The annotated three-dimensional training image dataset is based on the three-dimensional training image dataset. The position of the at least one landmark is annotated in the annotated three-dimensional training image dataset. The method also comprises a method step of training a function as a function of the three-dimensional training image dataset and of the annotated three-dimensional training image dataset. The method also comprises a method step of providing the trained function.

The three-dimensional (MRI) training image dataset is designed in particular like the above-described three-dimensional (MRI) image dataset.

In the method steps of receiving the three-dimensional training image dataset and of receiving the annotated three-dimensional training image dataset, the image datasets can be received from a database by a training interface. The database can be stored in particular on a server. In embodiments of the present invention, the database can be a PACS.

In particular, the position of the at least one landmark is annotated or drawn or marked in the annotated three-dimensional training image dataset. In particular, the annotated three-dimensional training image dataset can match the three-dimensional training image dataset, with the position of the at least one landmark being drawn or marked or annotated in the annotated three-dimensional training image dataset. In particular, the positions of more than one landmark can be annotated in the annotated three-dimensional training image dataset. Alternatively, the positions of the different landmarks can be annotated in copies of the three-dimensional training image dataset. In other words, the position of another landmark can be annotated in each copy.

In embodiments of the present invention, the position of the at least one landmark can be drawn or annotated in the annotated three-dimensional training image dataset in the form of one or more region(s). The different regions can indicate where the at least one landmark can be arranged in the annotated three-dimensional training image dataset. The different regions relate to different probabilities with which the at least one landmark is arranged inside the corresponding region.

In alternative embodiments of the present invention, the annotated three-dimensional training image dataset can comprise at least one three-dimensional coordinate. The three-dimensional coordinate indicates the position of the at least one landmark in the voxel matrix of the three-dimensional training image dataset.

In the method step of training, the function is applied to the three-dimensional training image dataset. The position of the at least one landmark is determined in the process. This position determined in such a way is compared with the position of the landmark in accordance with the annotated three-dimensional training image dataset. In the case of a deviation, the function is adjusted in such a way that the determined position lies closer to the position in accordance with the annotated three-dimensional training image dataset. This can be iteratively repeated until the two positions match or the deviation between the positions undershoots a threshold value. In other words, the deviation can be iteratively minimized by adjusting or training the function.

In particular, the method step of training can be repeated for a plurality of three-dimensional training image datasets and corresponding annotated three-dimensional training image datasets.

In particular, the function can be trained until a termination criterion is achieved. The termination criterion can be for example a maximum number of training cycles or iterations. Alternatively, the termination criterion can be a maximum number of three-dimensional training image datasets on which the function is trained. Alternatively, or in addition, the termination criterion can be an undershooting of a maximum deviation of the position determined with the function and the position of the at least one landmark specified in the annotated three-dimensional training image dataset.

In the method step of providing the trained function, the trained function is provided in such a way that it can be applied as described above to a three-dimensional image dataset.

The inventors have found that the function can be trained via supervised learning. The inventors have found that there is sufficient training data available. In particular, experience can be called upon in the case of the annotated three-dimensional training image datasets. In other words, there is already a large volume of (annotated) three-dimensional image datasets available, which can be used for training the function.

According to one aspect of embodiments of the present invention, the annotated three-dimensional training image dataset can be created by manually annotating the three-dimensional training image dataset.

In particular, the three-dimensional training image dataset can be annotated by an experienced medical operator. The medical operator can in particular be a medical-technical radiology assistant (acronym: MTRA) and/or a radiologist.

The medical operator can manually draw or annotate or mark a position of the at least one landmark in the three-dimensional training image dataset.

The medical operator can mark a voxel in the three-dimensional training image dataset in which the at least one landmark is mapped. In particular, the position of the at least one landmark or the annotated three-dimensional training image dataset can comprise the coordinates of the marked voxel.

In alternative embodiments of the present invention, the medical operator can draw one or more region(s) as a possible position of the at least one landmark in the three-dimensional training image dataset. The regions can indicate an uncertainty of the position of the at least one landmark. In other words, a region can indicate the region in the three-dimensional training image dataset in which the landmark is positioned with a particular probability. Different regions can relate to different probabilities. The corresponding region can become ever larger as the probability decreases.

In embodiments of the present invention, the three-dimensional training image dataset can be annotated several times by different medical operators. In particular, the annotated three-dimensional training image dataset can then be a mean of the different annotations. Alternatively, each of the annotated three-dimensional training image datasets can be used individually for training the function.

The inventors have found that the medical operator can manually annotate the three-dimensional training image dataset. In this way, the experience of the medical operator can be taken into account or used when training the function. In particular, a large number of annotated three-dimensional training image datasets can be produced or generated in this way.

One or more example embodiments of the present invention also relate to a determining system for determining an orientation of at least one diagnostically relevant sectional plane for heart imaging in a three-dimensional magnetic resonance imaging image dataset. The determining system comprises an interface and a computing unit. The interface is designed for providing the three-dimensional image dataset. The computing unit is designed for applying a trained function to the three-dimensional image dataset. A position of at least one landmark is determined. The computing unit is also designed for determining the orientation of the at least one diagnostically relevant sectional plane as a function of at least one landmark. The interface is also designed for providing the orientation of the at least one diagnostically relevant sectional plane.

A determining system of this kind can be designed in particular for carrying out the previously described method for determining an orientation of at least one diagnostically relevant sectional plane for heart imaging in a three-dimensional magnetic resonance imaging image dataset and its aspects. The determining system is designed to carry out this method and its aspects in that the interface and the computing unit are designed to carry out the corresponding method steps.

One or more example embodiments of the present invention also relate to a magnetic resonance imaging system. The magnetic resonance imaging system comprises an above-described determining system. The magnetic resonance imaging system is designed for recording the three-dimensional image dataset and/or a two-dimensional sectional image.

The MRI system preferably comprises a medical and/or diagnostic MRI system, which is configured and/or designed for recording medical and/or diagnostic MRI scans or image data or three-dimensional image datasets and/or two-dimensional sectional images, of an above-described examination object. The MRI system comprises a scanner unit for this purpose. The scanner unit of the MRI system preferably comprises a detector unit, in particular a magnetic unit, for recording the three-dimensional image dataset and/or the two-dimensional sectional image. Advantageously, the scanner unit, in particular the magnetic unit, comprises a base magnet, a gradient coil unit and a system coil or radio-frequency antenna unit. The system coil is permanently arranged inside the scanner unit and configured or designed for emitting an excitation pulse. For acquiring the magnetic resonance signal or radio signal or signal, the MRI system has at least one local coil, which can be arranged around a region to be examined of the examination object.

The base magnet of the scanner unit is designed for generating a homogeneous basic magnetic field with a defined and/or particular magnetic field strength, such as with a defined and/or particular magnetic field strength of 3 T or 1.5 T etc. In particular, the base magnet is designed for generating a strong, constant and homogeneous basic magnetic field. The homogeneous basic magnetic field is preferably arranged or can be found inside a scan region for the examination object of the MRI system. The gradient coil unit is designed for generating magnetic field gradients, which are used for spatial encoding during imaging.

The scan region is configured and/or designed for a scan of the examination object, in particular of the region to be examined of the examination object, for a medical MRI examination. For example, the scan region is cylindrical and/or cylindrically surrounded by the scanner unit for this purpose. For this purpose, the scanner unit has a housing of the housing unit at least partially surrounding the scan region. The housing surrounding the scan region can also be designed in one piece and/or formed in one piece with the side of the system coil of the scanner unit facing the scan region, or also separately from the system coil of the scanner unit.

A Field of View (FOV) and/or an isocenter of the MRI system is preferably arranged inside the scan region. The FOV preferably comprises a recording region of the MRI system, inside which the conditions for recording an MRI scan, in particular MRI image data, exist, such as a homogeneous basic magnetic field. The isocenter of the MRI system preferably comprises the region and/or point inside the MRI system, which has the optimum and/or ideal conditions for recording an MRI scan, in particular MRI image data. In particular, the isocenter comprises the most homogeneous basic magnetic field region inside the MRI system.

During an MRI examination the examination object is situated lying on a couch inside the scan region of the MRI system. By contrast, a medical operator or user is situated in a control room, which is separate from an examination room, in which the MRI system is arranged.

One or more example embodiments of the present invention also relate to a computer program product with a computer program and a computer-readable medium. An implementation largely in terms of software has the advantage that even previously used determining systems can be easily retrofitted by way of a software update in order to work in the described manner. Apart from the computer program, a computer program product of this kind can optionally comprise additional component parts, such as documentation and/or additional components, as well as hardware components, such as hardware keys (dongles, etc.), in order to use the software.

In particular, one or more example embodiments of the present invention also relate to a computer program product with a computer program, which can be loaded directly into a memory unit of a determining system, with program segments in order to carry out all steps of the above-described method for determining an orientation of at least one diagnostically relevant sectional plane for heart imaging in a three-dimensional magnetic resonance imaging image dataset and its aspects when the program segments are executed by the determining system.

In particular, one or more example embodiments of the present invention relate to a computer-readable storage medium on which program segments, which can be read and executed by a determining system, are stored in order to carry out all steps of the above-described method for determining an orientation of at least one diagnostically relevant sectional plane for heart imaging in a three-dimensional magnetic resonance imaging image dataset and its aspects when the program segments are executed by the determining system.

One or more example embodiments of the present invention also relate to a training system for providing a trained function for determining a position of at least one landmark in a three-dimensional magnetic resonance imaging image dataset. The training system comprises a training interface and a training computing unit. The training interface is designed for receiving at least one three-dimensional training image dataset. The training interface is also designed for receiving at least one annotated three-dimensional training image dataset. The annotated three-dimensional training image dataset is based on the three-dimensional training image dataset. The position of the at least one landmark is annotated in the annotated three-dimensional training image dataset. The training computing unit is designed for training a function as a function of the three-dimensional training image dataset and of the annotated three-dimensional training image dataset. The training interface is also designed for providing the trained function.

A training system of this kind can be designed in particular for carrying out the previously described method for determining a position of at least one landmark in a three-dimensional magnetic resonance imaging image dataset and its aspects. The training system is designed to carry out this method and its aspects in that the training interface and the training computing unit are designed to carry out the corresponding method steps.

One or more example embodiments of the present invention also relate to a training computer program product with a training computer program and a computer-readable training medium. An implementation largely in terms of software has the advantage, that even previously used training systems can be easily retrofitted by way of a software update in order to work in the described manner. Apart from the training computer program, a training computer program product of this kind can optionally comprise additional component parts, such as documentation and/or additional components, as well as hardware components, such as hardware keys (dongles, etc.), in order to use the software.

In particular, one or more example embodiments of the present invention also relate to a training computer program product with a training computer program, which can be loaded directly into a training memory unit of a training system, with training program segments in order to carry out all steps of the above-described method for determining a position of at least one landmark in a three-dimensional magnetic resonance imaging image dataset and its aspects when the training program segments are executed by the training system.

In particular, one or more example embodiments of the present invention relate to a computer-readable training storage medium on which training program segments, which can be read and executed by a training system, are stored in order to carry out all steps of the above-described method for determining a position of at least one landmark in a three-dimensional magnetic resonance imaging image dataset and its aspects when the program segments are executed by the training system.

### BRIEF DESCRIPTION OF THE DRAWINGS

The above-described properties, features and advantages of the present invention will become clearer and more comprehensible in connection with the following figures and their descriptions. The figures and descriptions are not intended to limit the present invention and its embodiments in any way.

Identical components are provided with corresponding reference numerals in different figures. As a rule, the figures are not to scale.

In the drawings:

FIG. 1 shows a first exemplary embodiment of a computer-implemented method for determining an orientation of at least one diagnostically relevant sectional plane for heart imaging in a three-dimensional magnetic resonance imaging image dataset,

FIG. 2 shows a second exemplary embodiment of a computer-implemented method for determining an orientation of at least one diagnostically relevant sectional plane for heart imaging in a three-dimensional magnetic resonance imaging image dataset,

FIG. 3 shows a third exemplary embodiment of a computer-implemented method for determining an orientation of at least one diagnostically relevant sectional plane for heart imaging in a three-dimensional magnetic resonance imaging image dataset,

FIG. 4 shows a fourth exemplary embodiment of a computer-implemented method for determining an orientation of at least one diagnostically relevant sectional plane for heart imaging in a three-dimensional magnetic resonance imaging image dataset,

FIG. 5 shows an exemplary embodiment of a computer-implemented method for providing a trained function for determining a position of at least one landmark in a three-dimensional magnetic resonance imaging image dataset,

FIG. 6 shows a determining system for determining an orientation of at least one diagnostically relevant sectional plane for heart imaging in a three-dimensional magnetic resonance imaging image dataset,

FIG. 7 shows a training system for providing a trained function for determining a position of at least one landmark in a three-dimensional magnetic resonance imaging image dataset,

FIG. 8 shows a magnetic resonance imaging system.

### DETAILED DESCRIPTION

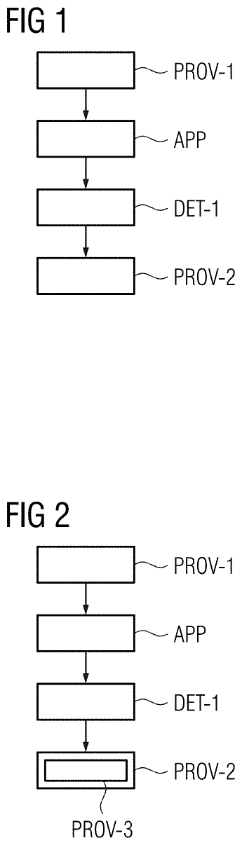

FIG. 1 shows a first exemplary embodiment of a computer-implemented method for determining an orientation of at least one diagnostically relevant sectional plane for heart imaging in a three-dimensional magnetic resonance imaging image dataset.

The three-dimensional magnetic resonance imaging (acronym: MRI) image dataset or three-dimensional image dataset comprises a three-dimensional representation of at least one part of an examination object. The examination object is a person, in particular a patient, or an animal. In particular, the represented part of the examination object comprises the heart or at least one part of the heart of the examination object. In other words, the three-dimensional image dataset comprises a three-dimensional representation of at least one part of the heart of the examination object.

The three-dimensional image dataset comprises a plurality of voxels, which are arranged in a three-dimensional voxel matrix. Each Voxel comprises a voxel value.

In embodiments of the present invention, the three-dimensional image dataset can comprise an overview scan of at least the part of the heart. The overview scan can have been recorded with an MRI system described in FIG. 7. In an overview scan an image quality, in particular a spatial resolution and/or a signal-to-noise ratio, is reduced in favor of a reduced scanning time.

In embodiments of the present invention, the three-dimensional image dataset can comprise a two-dimensional multi-slice scan. The two-dimensional multi-slice scan comprises a plurality of two-dimensional sectional images or slice images of the heart or of the part of the heart. The plurality of slice images is oriented in particular parallel among themselves or to each other. These slice images can be arranged spatially behind each other or be “stacked”. A “pseudo” three-dimensional representation of the heart or of the part of the heart is generated in this way. The individual slice images sample the heart or the part of the heart perpendicular to the image plane of the sectional images.

A sectional plane is a two-dimensional section through the three-dimensional image dataset. The two-dimensional section can be mapped or represented in a sectional image of the sectional plane. The sectional image can be recorded with an MRI system described in FIG. 7. The diagnostically relevant sectional plane describes in particular a predefined or standardized sectional plane through the heart. In other words, a predefined or standardized representation of a two-dimensional section through the heart is mapped or represented in the sectional image of the diagnostically relevant sectional plane. The predefined or standardized representation indicates how and in what connection which anatomical structures of the heart should be represented. A diagnosis and/or an examination of the examination object can be made or carried out using the representation of the diagnostically relevant sectional plane in a sectional image.

The orientation of the diagnostically relevant sectional plane describes the orientation relative to the examination object positioned in a scan region of the MRI system or relative to the MRI system.

In particular, the orientation describes a spatial situation or spatial orientation or angulation of the diagnostically relevant sectional plane in the space. The spatial situation can be described for example by a direction of a normal vector on the diagnostically relevant sectional plane. In embodiments, the orientation can also describe a position of the diagnostically relevant sectional plane relative to the examination object or the MRI system. The position can be specified for example by points, which should lie inside the diagnostically relevant sectional plane. In other words, the orientation can comprise information about the spatial situation and in embodiments of the present invention, about the position of the diagnostically relevant sectional plane.

In a method step of providing PROV-1, the three-dimensional image dataset is provided in particular by an interface SYS.IF. Providing PROV-1 can comprise retrieving or receiving or recording the three-dimensional image dataset.

In particular, the three-dimensional image dataset can be recorded with the MRI system. In embodiments, the three-dimensional image dataset can be provided by the MRI system.

Alternatively, the three-dimensional image dataset can be retrieved or received from a server, in particular from a database on the server. The server can in particular be a local server or a Cloud server. The database can in particular be a picture archiving and communication system (acronym: PACS).

In a method step of applying APP a trained function, the trained function is applied to the three-dimensional image dataset. In other words, the input data of the trained function comprises the three-dimensional image dataset. A position of at least one landmark is determined in the process.

The trained function can be based on a neural convolutional network (acronym: CNN) and/or a U-Network (U-net).

In embodiments of the present invention, in each case the position of more than one landmark can be determined in the three-dimensional image dataset.

The at least one landmark relates to an anatomy of the heart. The at least one landmark can be for example one of the following landmarks: apex, mitral valve, aortic valve, pulmonary valve, tricuspid valve.

In embodiments of the present invention, the at least one landmark can be one of the following landmarks:

left ventricular apex (acronym: LV apex)
right ventricular apex (acronym: RV apex)
right ventricle extent (acronym: RV extent)
mitral valve, midpoint (acronym: MV midpoint)
aortic valve, midpoint (acronym: AV midpoint)
tricuspid valve, midpoint (acronym: TV midpoint)
pulmonary valve, midpoint (acronym: PA center point)
aortic arch
descending aorta
four points on the vessel wall of the aortic bulb (anterior, posterior, left, right)
four points on the vessel wall of the pulmonary artery (left, right, superior, inferior) at a suitable point for a flow measurement of the pulmonary artery (pulmonary artery flow plane)
four points on the annulus of the pulmonary valve (left, right, superior, inferior) (pulmonary valve insertion)
four points on the annulus of the mitral valve (septal, lateral, superior, inferior) (mitral valve insertion)
four points on the annulus of the tricuspid valve (septal, lateral, superior, inferior) (tricuspid valve insertion)
right ventricle insertion points (anterior, posterior) on the left ventricle
descending aorta centerline
ascending aorta centerline.

The position of the at least one landmark can be described by its position in the voxel matrix of the three-dimensional image dataset. In other words, the position can indicate where in the voxel matrix the at least one landmark is mapped.

In embodiments of the present invention, the position of the at least one landmark can be indicated in the form of a probability distribution (heatmap). A probability can be assigned to each voxel of the voxel matrix of the three-dimensional image dataset, which indicates how probably the at least one landmark is mapped in the corresponding voxel.

In a method step of determining DET-1 the orientation of the at least one diagnostically relevant sectional plane, the orientation is determined as a function of at least one landmark.

In other words, the orientation is determined as a function of position of the at least one landmark. In particular, the orientation can be determined as a function of positions of more than one landmark. In particular, the orientation can be determined as a function of a selection of the particular positions of the landmarks. The selection can be predefined or specified for the diagnostically relevant sectional plane. Different selections of landmarks or their positions can be taken into account for different diagnostically relevant sectional planes. In other words, the orientation of different diagnostically relevant sectional planes can depend on different landmarks.

In particular, a spatial connection between the position of the at least one landmark and the diagnostically relevant sectional plane can be taken into account when determining DET-1 the orientation. In particular, the at least one landmark can lie in the diagnostically relevant sectional plane.

In particular, the probability distribution can be taken into account when determining DET-1 the orientation of the diagnostically relevant sectional plane. If, for example, three landmarks should lie in the diagnostically relevant sectional plane, the orientation of the diagnostically relevant sectional plane can be determined in such a way that the mean of the probabilities of the regions, through which the diagnostically relevant sectional plane runs, is maximal for all three landmarks.

In embodiments of the present invention, a prior knowledge in respect of the orientation can also be taken into account when determining DET-1 the orientation. The prior knowledge can take into account for example a known orientation relative to a standard orientation in the MRI system (coronal, sagittal, axial). Alternatively, or in addition, the prior knowledge can take into account a situation of the heart or the like.

The diagnostically relevant sectional plane can in particular be one of the following sectional planes: four chamber plane, three chamber plane, two chamber plane, vertical long axis, horizontal long axis, short axis.

A theoretical situation of said sectional planes is described for example in Maier et al., “Kardiale Magnetresonanztomographie—Anatomie and Planung”, Journal for Kardiologie—Austrian Journal of Cardiology 2003; 10: 3-7.

In embodiments of the present invention, the at least one diagnostically relevant sectional plane can be one of the following sectional planes:

vertical long axis (acronym: VLA)
horizontal long axis (acronym: HLA)
three chamber view (acronym: 3CH)
four chamber view (acronym: 4CH)
short axis (acronym: SAX)
right ventricle two chamber view (acronym: RV-2CH)
right ventricle three chamber view (acronym: RV-3CH)

### Aortic Flow Plane

left ventricular outflow tract plane (acronym: LVOT)
mitral valve plane (acronym: MV Plane).

The orientation of the sectional planes can be derived from or depend on the landmarks or their positions indicated below in brackets after the sectional planes:

VLA (LV apex, MV midpoint+prior knowledge: parasagittal)
HLA (LV apex, MV midpoint+prior knowledge: orthogonal to VLA)
3CH (LV apex, MV midpoint, AV midpoint)
4CH (LV apex, MV midpoint, RV extent)
SAX (midpoint of LV apex and MV midpoint, RV extent+prior knowledge: orthogonal to VLA and HLA)
RV-2CH (RV apex, TV midpoint+prior knowledge: parasagittal)
RV-3CH (RV apex, TV midpoint, PA center point)
Aortic Flow Plane (Aortic bulb (anterior), Aortic bulb (posterior), Aortic bulb (left), Aortic bulb (right))
LVOT (Aortic bulb (anterior), Aortic bulb (posterior)+prior knowledge: slice in the midpoint of the two landmarks, orthogonal to the connecting vector of the landmarks)
MV Plane (MV insertion (septal), MV insertion (lateral), MV insertion (superior), MV insertion (inferior)).

In a method step of providing PROV-2 the orientation of the at least one diagnostically relevant sectional plane, the orientation is provided in particular by an interface SYS.IF. In particular, the orientation can be provided in the form of a vector in respect of a coordinate system of the MRI system. Alternatively, the orientation can be provided as the orientation of a magnetic field gradient, which is designed to orient the protons in the diagnostically relevant sectional plane in such a way that they can be excited with an excitation pulse.

FIG. 2 shows a second exemplary embodiment of a computer-implemented method for determining an orientation of at least one diagnostically relevant sectional plane for heart imaging in a three-dimensional magnetic resonance imaging image dataset.

The first exemplary embodiment described in respect of FIG. 1 is developed in the second exemplary embodiment described here with a method step of providing PROV-3 at least one first scanning parameter value. The other method steps are designed in accordance with the description relating to FIG. 1.

The at least one first scanning parameter value is designed for controlling a medical-technical MRI system 10 for recording a two-dimensional sectional image of the at least one diagnostically relevant sectional plane.

In particular, the first scanning parameter value is designed in such a way that a gradient control unit 19 of an MRI system 10 can actuate a gradient coil unit 18 of the MRI system 10 via the first scanning parameter value. The MRI system 10 is illustrated in the description relating to FIG. 7.

In particular, the first scanning parameter value can be designed in such a way that the gradient coil unit 18 generates a magnetic field gradient, which is oriented perpendicular to the diagnostically relevant sectional plane. In other words, the first scanning parameter value can specify a direction of the magnetic field gradient. The magnetic field gradient, together with a homogeneous basic magnetic field, which is generated by a base magnet 16, forms a magnetic field in which the examination object is arranged.

In particular, the gradient coil unit 18 is actuated with the first scanning parameter value in such a way that the proton spins incorporated by the examination object precess or rotate in the diagnostically relevant sectional plane in a particular Larmor frequency. The Larmor frequency is proportional to a magnetic field strength of the magnetic field. In particular, the first scanning parameter value can thus specify a strength of the magnetic field gradient. In particular, the magnetic field gradient inside the diagnostically relevant sectional plane is constant. In particular, all spins in the diagnostically relevant sectional plane then precess at the same Larmor frequency. In particular, the spins in the diagnostically relevant sectional plane can then be excited with an excitation pulse of exactly this frequency. The signal, which the spins emit in the magnetic field on relaxation, can then be acquired. The two-dimensional sectional image of the diagnostically relevant sectional plane can be reconstructed from this signal. The excited spins are arranged perpendicular to the magnetic field gradient in a slice in the examination object. The slice is arranged in particular in the diagnostically relevant sectional plane.

In embodiments of the present invention, the first scanning parameter value can specify a “steepness” of the magnetic field gradient. The thickness of the slice of the excited spins perpendicular to the diagnostically relevant sectional plane can be specified by the “steepness” of the magnetic field gradient. The flatter the magnetic field gradient, the more spins precess at least approximately at the frequency of the excitation pulse and are thus excited, the thicker the slice is therefore. The steeper the magnetic field gradient, the fewer spins precess at the frequency of the excitation pulse and the thinner the excited slice is therefore.

The at least one first scanning parameter value is derived in the method step of providing PROV-3 in particular from the orientation of the diagnostically relevant sectional plane. In particular, the first scanning parameter value is designed in such a way that a magnetic field gradient can be generated by the first scanning parameter value, which excites spins inside the diagnostically relevant sectional plane. In other words, the generated magnetic field gradient is then oriented perpendicular to the diagnostically relevant sectional plane or parallel to a normal vector on the diagnostically relevant sectional plane.

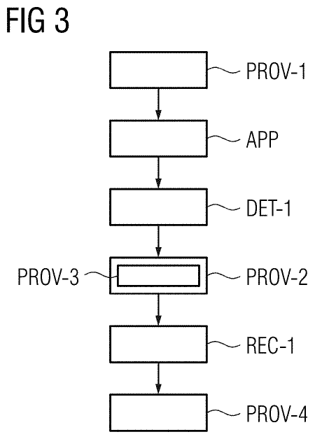

FIG. 3 shows a third exemplary embodiment of a computer-implemented method for determining an orientation of at least one diagnostically relevant sectional plane for heart imaging in a three-dimensional magnetic resonance imaging image dataset.

The second exemplary embodiment described in respect of FIG. 2 is developed in the third exemplary embodiment described here with a method step of recording REC-1 a two-dimensional sectional image and providing PROV-4 the two-dimensional sectional image. The other method steps are designed in accordance with the description relating to FIGS. 1 and 2.

In the method step of the recording REC-1 the two-dimensional sectional image, the two-dimensional sectional image is recorded with the MRI system 10 as a function of the first scanning parameter value. The basic principle of the MRI system 10 is illustrated in the description relating to FIG. 7.

The method step of recording REC-1 can comprise in particular a method step of receiving or reading out a signal and a method step of reconstructing the two-dimensional sectional image from the signal.

The signal can be received in particular on relaxation of the spins in the magnetic field. The signal can be received with an antenna, in particular a coil. The antenna can be a system coil 20 or a local coil 38.

In the method step of reconstructing, the two-dimensional sectional image is reconstructed on the basis of the received signal. In particular, the reconstruction can be based on a filtered backpropagation.

In the method step of providing PROV-4 the two-dimensional sectional image, the sectional image is provided in particular by an interface SYS.IF.

In embodiments of the present invention, a medical operator can be provided with the sectional image. For this the sectional image can be displayed on a display unit, in particular a monitor or a screen.

Alternatively, or in addition, the two-dimensional sectional image can be stored on a server, in particular in a database. The server can be a local server or a Cloud server. In particular, the database can be a PACS.

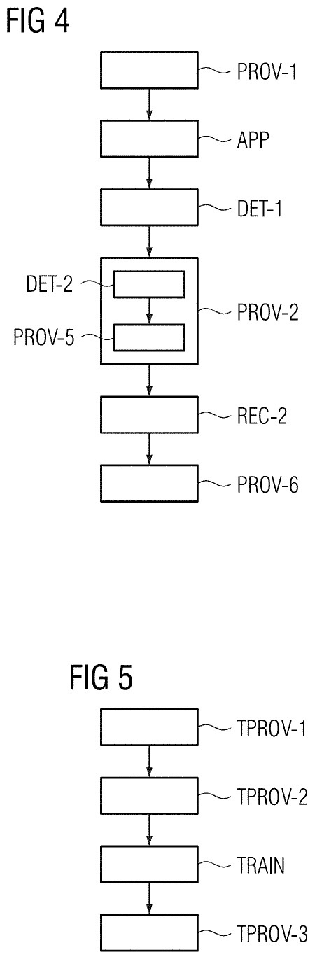

FIG. 4 shows a fourth exemplary embodiment of a computer-implemented method for determining an orientation of at least one diagnostically relevant sectional plane for heart imaging in a three-dimensional magnetic resonance imaging image dataset.

The first exemplary embodiment described in respect of FIG. 1 is developed in the fourth exemplary embodiment described here with the method steps of determining DET-2 an extent of a three-dimensional volume image perpendicular to the diagnostically relevant sectional plane, providing PROV-5 at least one second scanning parameter value, recording REC-2 the three-dimensional volume image and providing PROV-6 the three-dimensional volume image. The other method steps are designed in accordance with the description relating to FIG. 1.

The three-dimensional volume image is a three-dimensional representation or mapping of the heart or of the part of the heart. The heart in the diagnostically relevant sectional plane is also mapped in the three-dimensional volume image. The size of the three-dimensional volume image is specified or spanned by the diagnostically relevant sectional plane and an extent of the three-dimensional volume image perpendicular to the diagnostically relevant sectional plane. The diagnostically relevant sectional plane can lie at any position of the three-dimensional volume image. In particular, the diagnostically relevant sectional plane can form a side face of the three-dimensional volume image. Alternatively, the diagnostically relevant sectional plane can be arranged for example in the center of the three-dimensional volume image.

In the method step of determining DET-2 the extent of the three-dimensional volume image, the extent thereof perpendicular to the diagnostically relevant sectional plane is determined. In particular, the extent can be determined in such a way that an anatomy of the heart is mapped in the three-dimensional volume image. In other words, the extent can be determined as a function of an anatomical actuality of the heart. The anatomy or the anatomical actuality can be determined as a function of the three-dimensional image dataset. The anatomical actuality can depend for example on one or more landmark(s). For example, the extent can be designed in such a way that an aortic arch of the heart is mapped in the volume image. The extent can be indicated in particular in a metric unit. For example, the extent can comprise 0.1 cm or 0.5 cm or 1 cm or 2 cm.

In the method step of providing PROV-5, the at least one second scanning parameter value for controlling the MRI system when recording the three-dimensional volume image is provided. The at least one second scanning parameter value is derived from the orientation of the diagnostically relevant sectional plane and the extent. In particular, the at least one second scanning parameter value is designed in such a way that with it, the MRI system can be actuated in such a way that the third volume image can be recorded. The at least one second scanning parameter value can be designed at least partially as described in respect of the first scanning parameter value in the description relating to FIG. 2. In addition, the at least one second scanning parameter value can comprise information about how a strength of the magnetic field gradient has to be varied so, with a constant excitation pulse, the spins in the entire region of the heart incorporated by the volume image can be iteratively excited and the corresponding signal can be acquired. In other words, the at least one second scanning parameter value can comprise a sequence of strengths of the magnetic field gradient. Alternatively, the at least one second scanning parameter value can comprise information about a change in the frequency of the excitation pulse so, with a constant magnetic field gradient, due to the change in the frequency of the excitation pulse, the spins in the entire region of the region mapped in the volume image are excited. In other words, the at least one second scanning parameter value can comprise a sequence of frequencies of the excitation pulse.

In the method step of recording REC-2 the three-dimensional volume image, the three-dimensional volume image is recorded with the MRI system. The MRI system is actuated with the at least one second excitation pulse. Recording REC-2 of the three-dimensional volume image can take place analogously to the recording REC-1 of the two-dimensional sectional image described in the description relating to FIG. 3. In contrast thereto, the frequency of the excitation pulse and/or the strength of the magnetic field gradient is varied during the recording in order to excite the spins in the entire region mapped in the volume image.

In the method step of providing PROV-6 the three-dimensional volume image, the three-dimensional volume image is provided in particular via an interface SYS.IF. In particular, the three-dimensional volume image is displayed to a medical operator. Alternatively, or in addition, the three-dimensional volume image can be stored in a database or on a server. Providing PROV-6 the three-dimensional volume image can take place analogously to providing PROV-4 the two-dimensional sectional image in accordance with the description relating to FIG. 3.

FIG. 5 shows an exemplary embodiment of a computer-implemented method for providing a trained function for determining a position of at least one landmark in a three-dimensional magnetic resonance imaging image dataset.

The three-dimensional image dataset and the at least one landmark are designed in particular as described above in respect of FIGS. 1 to 4.

The trained function is designed for application in the method step of applying APP the trained function in accordance with the description relating to FIG. 1.

In other words, the input data of the trained function comprises the three-dimensional image data and the output data the position of the at least one landmark.

The method for training the trained function will be described below.

In a method step of receiving TPROV-1 at least one three-dimensional training image dataset, the at least one three-dimensional training image dataset is received via a training interface TSYS.IF. The three-dimensional training image dataset can be designed analogously to the three-dimensional image dataset in accordance with the description relating to FIG. 1. In particular, the three-dimensional training image dataset can be received from a server, in particular from a database on the server. The server can be a local server or a Cloud server. The database can be for example a PACS.

In particular, a plurality of three-dimensional training image datasets can be received.

In a method step of receiving TPROV-2 at least one annotated three-dimensional training image dataset, the at least one annotated three-dimensional training image dataset is received by the training interface TSYS.IF. The annotated three-dimensional training image dataset can be received from the server, in particular from the database on the server.

If a plurality of three-dimensional training image datasets is received in the method step of receiving TPROV-1 at least one three-dimensional training image dataset, at least one annotated three-dimensional training image dataset is received for each of the three-dimensional training image datasets.

The annotated three-dimensional training image dataset is based on the three-dimensional training image dataset. In particular, the position of the at least one landmark is annotated or marked or drawn or identified in the annotated three-dimensional training image dataset. In other words, the annotated three-dimensional training image dataset comprises the position of the at least one landmark in the three-dimensional training image dataset.

In particular, the position of the at least one landmark can indicate the voxel in the three-dimensional training image dataset, which maps the at least one landmark. In particular, the annotated three-dimensional training image dataset can then comprise a coordinate of the voxel in the voxel matrix.

Alternatively, the position of the at least one landmark can indicate one or more region(s) in the three-dimensional training image dataset. A probability, with which the at least one landmark is mapped inside the corresponding region, can be assigned to each region. In particular, the annotated three-dimensional training image dataset can then match a voxel matrix in accordance with the voxel matrix of the three-dimensional training image dataset, which is divided into one or more region(s).

In particular, the position of more than one landmark can be annotated in the annotated three-dimensional training image dataset. Alternatively, an annotated three-dimensional training image dataset can be received for a three-dimensional training image dataset for each landmark. The position of another landmark can be annotated in each annotated training image dataset.

In particular, the annotated three-dimensional training image dataset can be manually annotated. In other words, the position of the at least one landmark can be manually drawn or annotated or marked by a medical operator. In particular, the medical operator can mark the voxel, which maps the at least one landmark. Alternatively, the medical operator can draw the region(s) described above as the position of the at least one landmark.

In a method step of training a function, the function is trained as a function of the three-dimensional training image dataset and as a function of the annotated three-dimensional training image dataset.

For this the function is applied to the at least one three-dimensional training image dataset. A position of the at least one landmark is determined in the three-dimensional training image dataset. The position determined in such a way is compared with the position in the annotated three-dimensional training image dataset. If the two positions differ from each other the function is adjusted or modified in such a way that on renewed application of the function to the three-dimensional training image dataset, the determined position better matches the position in accordance with the annotated three-dimensional training image dataset. This adjusting describes the training of the function.

In particular, this method can be repeated for the plurality of three-dimensional training image datasets and the corresponding plurality of annotated three-dimensional training image datasets.

In particular, the function can be iteratively adjusted further until a termination criterion is attained. The termination criterion can be for example a maximum number of iterations and/or a maximum number of three-dimensional training image datasets and/or an undershooting of a maximum deviation of the determined position and the position in accordance with the annotated three-dimensional training image dataset.

The function can be trained separately for the different landmarks. Alternatively, the function can be trained for all landmarks at once. In particular, the function can be trained for all landmarks at once if the landmarks are all annotated in an annotated three-dimensional training image dataset.

In a method step of providing TPROV-3 the trained function, the trained function is provided, in particular via the training interface TSYS-IF. In particular, the trained function is provided for application in the method in accordance with one of FIGS. 1 to 3. In particular, the trained function can be provided for use on a computing unit. In particular, the trained function can be stored or saved on a memory unit in the method step of providing TPROV-3 the trained function.

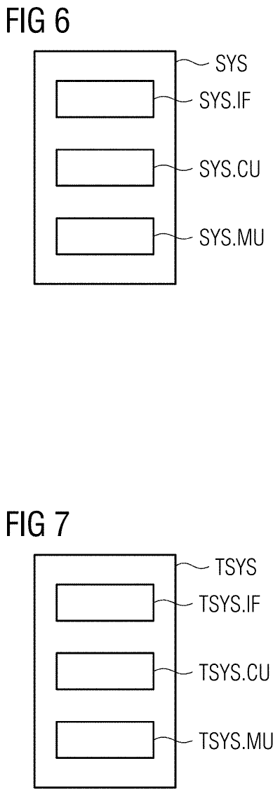

FIG. 6 shows a determining system SYS for determining an orientation of at least one diagnostically relevant sectional plane for heart imaging in three-dimensional magnetic resonance imaging image dataset, FIG. 7 shows a training system TSYS for providing a trained function for determining a position of at least one landmark in a three-dimensional magnetic resonance imaging image dataset.

The represented determining system SYS for determining an orientation of at least one diagnostically relevant sectional plane for heart imaging in a three-dimensional MRI image dataset is designed to carry out an inventive method for determining an orientation of at least one diagnostically relevant sectional plane for heart imaging in a three-dimensional MRI image dataset. The represented training system TSYS is designed to carry out an inventive method for providing the trained function for determining a position of at least one landmark in a three-dimensional magnetic resonance imaging image dataset. The determining system SYS comprises an interface SYS.IF, a computing unit SYS.CU and a memory unit SYS.MU. The training system TSYS comprises a training interface TSYS.IF, a training computing unit TSYS.CU and a training memory unit TSYS.MU.

The determining system SYS and/or the training system TSYS can in particular be a computer, a microcontroller or an integrated circuit (IC). Alternatively, the determining system SYS and/or the training system TSYS can be a real or virtual computer network (a technical name for a real computer network is “Cluster”, a technical name for a virtual computer network is “Cloud”). The determining system SYS and/or the training system TSYS can be designed as a virtual system, which is implemented on a computer or a real computer network or a virtual computer network (a technical name is “virtualization”).

The interface SYS.IF and/or the training interface TSYS.IF can be a hardware or software interface (for example a PCI bus, USB or Firewire). The computing unit SYS.CU and/or the training computing unit TSYS.CU can comprise hardware and/or software component parts, for example a microprocessor or what is known as an FPGA (Field Programmable Gate Way). The memory unit SYS.MU and/or the training memory unit TSYS.MU can be designed as a Random Access Memory (RAM) or as a permanent mass storage (hard disk, USB stick, SD card, Solid State Disk (SSD)).

The interface SYS.IF and/or the training interface TSYS.IF can in particular comprise a plurality of sub-interfaces, which carry out different method steps of the respective inventive method. In other words, the interface SYS.IF and/or the training interface TSYS.IF can be designed as a plurality of interfaces SYS.IF and/or training interfaces TSYS.IF. The computing unit SYS.CU and/or the training computing unit TSYS.CU can in particular comprise a plurality of sub-computing units, which carry out different method steps of the respective inventive method. In other words, the computing unit SYS.CU and/or the training computing unit TSYS.CU can be designed as a plurality of computing units SYS.CU and/or training computing units TSYS.CU.

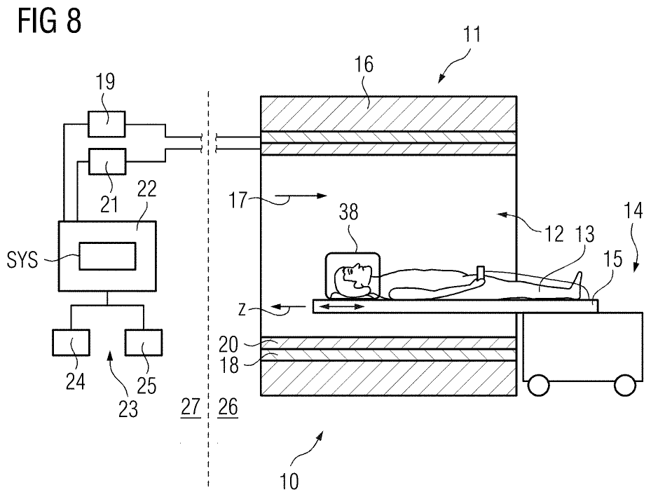

FIG. 8 shows a magnetic resonance imaging system 10.

The MRI system 10 is schematically represented. The MRI system 10 comprises a scanner unit 11 formed by a magnetic unit. In addition, the MRI system 10 has a scan region 12 for scanning an examination object 13, in particular a patient. In the present exemplary embodiment, the scan region 12 is cylindrical and cylindrically surrounded in a circumferential direction by the scanner unit 11, in particular by the magnetic unit. Basically, a design of the scan region 12 that differs herefrom is always conceivable, however. The examination object 13 can be pushed and/or moved via a positioning apparatus 14 of the MRI system 10 into the scan region 12. The positioning apparatus 14 has for this purpose a couch 15 configured to move inside the scan region 12. In particular, the couch 15 is mounted to be movable in the direction of a longitudinal extension of the scan region 12 and/or in the z-direction.

The scanner unit 11, in particular the magnetic unit, comprises a superconducting base magnet 16 for generating a strong and, in particular, constant basic magnetic field 17. Furthermore, the scanner unit 11, in particular the magnetic unit, has a gradient coil unit 18 for generating magnetic field gradients, which are used for spatial encoding during imaging. The gradient coil unit 18 is controlled via a gradient control unit 19 of the MRI system 10. In particular, an orientation of the magnetic field gradient can be controlled with the gradient control unit 19. In particular, the gradient control unit 19 can control the orientation of the magnetic field gradient with a first scanning parameter value. The scanner unit 11, in particular the magnetic unit, also comprises a radio-frequency antenna unit or a system coil 20 for exciting a polarization, which establishes itself in the basic magnetic field 17 generated by the base magnet 16. The system coil 20 is permanently arranged inside the scanner unit 11, in particular the magnetic unit. The system coil 20 is controlled by a system coil control unit 21 of the MRI system 10 and irradiates radio-frequency magnetic resonance sequences into the scan region 12 of the magnetic resonance apparatus 10.

For acquiring magnetic resonance signals or signals, the MRI system 10 has at least one local radio-frequency coil or local coil 38, which can be positioned around a region to be examined of the examination object 13. A local coil 38 is selected for the pending magnetic resonance examination as a function of the region to be examined of the examination object 13. For example, a local head radio-frequency coil for a head examination or a local knee radio-frequency coil for a knee examination, etc. The local coil 38 comprises at least one above-described coil element. In particular, if the local coil 38 comprises more than one coil element, the coil elements of the local coil 38 can be individually read out or actuated.

The MRI system 10 has a system control unit 22 for controlling the base magnet 16, the gradient control unit 19 and for controlling the system coil control unit 21. The system control unit 22 centrally controls the MRI system 10, such as carrying out a predetermined imaging gradient echo sequence. In addition, the system control unit 22 comprises an evaluation unit (not represented in more detail) for evaluation of MRI scans or medical image data, which are/is recorded during the magnetic resonance examination.

Furthermore, the MRI system 10 comprises a user interface 23, which is connected to the system control unit 22. Control information, such as imaging parameters, and reconstructed MRI scans can be displayed for a user or a medical operator on a display unit 24, for example on at least one monitor, of the user interface 23. Furthermore, the user interface 23 has an input unit 25 via which information and/or parameters can be input by the user during a measuring process.

The scanner unit 11 of the MRI system 10, together with the positioning apparatus 14, is arranged inside an examination room 26. By contrast, the system control unit 22, together with the user interface 23, is arranged inside a control room 27. The control room 27 is separate from the examination room 26. In particular, the examination room 26 is shielded in respect of radio-frequency radiation from the control room 27. During a magnetic resonance examination or recording of an MRI scan, the examination object 13 is located inside the examination room 26, whereas the user is located inside the control room 27.

The represented MRI system 10 can of course comprise further components, which MRI systems 10 conventionally have. A general mode of operation of the MRI system 10 is known to a person skilled in the art, moreover, so a detailed description of the further components will be omitted.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

Where it has not yet expressly occurred, but expedient and within the meaning of the present invention, individual exemplary embodiments, individual partial aspects or features thereof can be combined with each other or interchanged without departing from the scope of the present invention. Advantages of the present invention described with reference to one exemplary embodiment also relate without being expressly mentioned, where transferrable, to other exemplary embodiments.

What is claimed is:

1. A computer-implemented method for determining an orientation of at least one diagnostically relevant sectional plane for heart imaging in a three-dimensional magnetic resonance imaging image dataset, the computer-implemented method comprising:
providing the three-dimensional magnetic resonance imaging image dataset;
applying a trained function to the three-dimensional magnetic resonance imaging image dataset to determine a position of at least one landmark;
determining the orientation of the at least one diagnostically relevant sectional plane as a function of the at least one landmark; and
providing the orientation of the at least one diagnostically relevant sectional plane.

2. The computer-implemented method as claimed in claim 1,
wherein the three-dimensional magnetic resonance imaging image dataset maps at least one part of a heart, and
wherein the three-dimensional magnetic resonance imaging image dataset is an overview scan of the at least one part of the heart.

3. The computer-implemented method as claimed in claim 2,
wherein the at least one landmark is one of an apex, a mitral valve, an aortic valve, a pulmonary valve, or a tricuspid valve.

4. The computer-implemented method as claimed in claim 2,
wherein the at least one diagnostically relevant sectional plane is one of a four chamber plane, a three chamber plane, a two chamber plane, a vertical long axis, a horizontal long axis, or a short axis.

5. The computer-implemented method as claimed in claim 1, wherein the applying of the trained function comprises:
determining the position of the at least one landmark in the three-dimensional magnetic resonance imaging image dataset in the form of a probability distribution for the position of the at least one landmark.

6. The computer-implemented method as claimed in claim 1, wherein the providing of the orientation of the at least one diagnostically relevant sectional plane comprises:
providing at least one first scanning parameter value for controlling a magnetic resonance imaging system for recording a two-dimensional sectional image of the at least one diagnostically relevant sectional plane.

7. The computer-implemented method as claimed in claim 6, wherein the at least one first scanning parameter value is derived from the orientation of the at least one diagnostically relevant sectional plane.

8. The computer-implemented method as claimed in claim 6, further comprising:
recording the two-dimensional sectional image with the magnetic resonance imaging system as a function of the at least one first scanning parameter value; and
providing the two-dimensional sectional image.

9. The computer-implemented method as claimed in claim 1, further comprising:
determining an extent of a three-dimensional volume image perpendicular to the at least one diagnostically relevant sectional plane, wherein the three-dimensional volume image is spanned by the diagnostically relevant sectional plane and the extent;
providing at least one scanning parameter value for controlling a magnetic resonance imaging system for recording the three-dimensional volume image, wherein the at least one scanning parameter value is derived from the orientation of the at least one diagnostically relevant sectional plane and the extent;
recording the three-dimensional volume image with the magnetic resonance imaging system as a function of the at least one scanning parameter value; and
providing the three-dimensional volume image.

10. The computer-implemented method as claimed in claim 1, wherein the trained function is based on at least one of a neural convolutional network or a U-Network.

11. A computer-implemented method for providing a trained function for determining a position of at least one landmark in a three-dimensional magnetic resonance imaging image dataset, the computer-implemented method comprising:
receiving at least one three-dimensional training image dataset;
receiving at least one annotated three-dimensional training image dataset, wherein the at least one annotated three-dimensional training image dataset is based on the at least one three-dimensional training image dataset, and wherein the position of the at least one landmark is annotated in the at least one annotated three-dimensional training image dataset;
training a function as a function of the at least one three-dimensional training image dataset and the at least one annotated three-dimensional training image dataset; and
providing the trained function.

12. The computer-implemented method as claimed in claim 11, further comprising:
manually annotating the at least one three-dimensional training image dataset to create the at least one annotated three-dimensional training image dataset.

13. A determining system to determine an orientation of at least one diagnostically relevant sectional plane for heart imaging in a three-dimensional magnetic resonance imaging image dataset, the determining system comprising:
an interface configured to
provide the three-dimensional magnetic resonance imaging image dataset, and
provide an orientation of the at least one diagnostically relevant sectional plane; and
at least one processor configured to
apply a trained function to the three-dimensional magnetic resonance imaging image dataset to determine a position of at least one landmark, and
determine the orientation of the at least one diagnostically relevant sectional plane as a function of at least one landmark.

14. A magnetic resonance imaging system comprising:
the determining system as claimed in claim 13, wherein
the magnetic resonance imaging system is configured to acquire at least one of the three-dimensional magnetic resonance imaging image dataset or a two-dimensional sectional image.

15. A non-transitory computer program product including a computer program, which is loadable into a memory of a determining system, the computer program including program segments that, when executed by the determining system, cause the determining system to perform the computer-implemented method as claimed in claim 1.

16. A non-transitory computer-readable storage medium storing program segments that, when executed by a determining system, cause the determining system to perform the computer-implemented method of claim 1.

17. The computer-implemented method as claimed in claim 2, wherein the applying of the trained function comprises:
determining the position of the at least one landmark in the three-dimensional magnetic resonance imaging image dataset in the form of a probability distribution for the position of the at least one landmark.

18. The computer-implemented method as claimed in claim 7, further comprising:
recording the two-dimensional sectional image with the magnetic resonance imaging system as a function of the at least one first scanning parameter value; and
providing the two-dimensional sectional image.

19. The computer-implemented method as claimed in claim 5, further comprising:
determining an extent of a three-dimensional volume image perpendicular to the at least one diagnostically relevant sectional plane, wherein the three-dimensional volume image is spanned by the diagnostically relevant sectional plane and the extent;
providing at least one scanning parameter value for controlling a magnetic resonance imaging system for recording the three-dimensional volume image, wherein the at least one scanning parameter value is derived from the orientation of the at least one diagnostically relevant sectional plane and the extent;
recording the three-dimensional volume image with the magnetic resonance imaging system as a function of the at least one scanning parameter value; and
providing the three-dimensional volume image.

20. The computer-implemented method as claimed in claim 9, wherein the trained function is based on at least one of a neural convolutional network or a U-Network.
